(19) United States
(12) Patent Application Publication
(10) Pub. No.: US 2022/0035233 A1
(43) Pub. Date: Feb. 3, 2022

(54) FILM ASSEMBLY

(71) Applicant: Michael Leaf, Chula Vista, CA (US)

(72) Inventor: Michael Leaf, Chula Vista, CA (US)

(21) Appl. No.: 16/942,453

(22) Filed: Jul. 29, 2020

(51) Int. Cl. G03C 1/805; B32B 3/08; B32B 7/12; A61K 8/02; A61Q 1/02

(52) U.S. Cl. CPC G03C 1/805; B32B 3/08; B32B 7/12; A61K 8/0204; A61Q 1/025; B44C 1/1758; B32B 2554/00; B32B 2429/00; B32B 2451/00; B32B 2405/00; B32B 2307/748

(57) ABSTRACT

A film assembly for transferring an image to a surface includes a base layer, an ink layer that is removably adhered to the base layer, a developer layer that develops the ink layer and a transfer layer that is removably adhered to the developer layer, the transfer layer including the image that is configured to be transferred onto the surface. The base layer and the rear layer can be adhered together to form a rear layer. A frame layer can be removably adhered to the transfer layer. The ink layer can contain a non-toxic ink. The transfer layer can be manufactured with resilient materials. The frame layer can be removed from the transfer layer. The rear layer can be removed from the developer layer. The film assembly can be within a film assembly array of a plurality of film assemblies and can be disposed within a film assembly housing.

### BACKGROUND

The practice of applying images to surfaces upon contact has a few forms known in the art. Decals are images or patterns that are applied to surfaces using adhesives, heat, water, or other application aids. Modernly, decals are designed using computer software and sent to machines to print or cut the decals out of sheet materials. Other forms of images applied to surfaces include temporary tattoos. These non-permanent images may also be printed to adhesive sheet materials, but may also be drawn, painted, or airbrushed. Like decals, these images can be designed using computer software and printed to sticker adhesives that allow the images to be applied to human skin. While there have been numerous methods of applying images to surfaces, as mentioned above, these types of applied surface images need to be previously designed or created by an artist.

### SUMMARY

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

The present invention is directed toward a film assembly for transferring an image to a surface. In various embodiments, the film assembly can include a base layer, an ink layer, a developer layer, and a transfer layer.

In certain embodiments, the ink layer can be removably adhered to the base layer.

In some embodiments, the developer layer can develop the ink layer.

In various embodiments, the transfer layer can be removably adhered to the developer layer and the transfer layer can include the image that is configured to be transferred onto the surface.

In certain embodiments, the base layer and the ink layer can be adhered together to form a rear layer.

In some embodiments, the rear layer can have an adhesive side and an opposed non-adhesive side.

In certain embodiments, the rear layer can be removable from the developer layer.

In various embodiments, the transfer layer can include a frame layer with a frame layer base portion, the frame layer can be removably adhered to the transfer layer.

In some embodiments, the frame layer can include a removable base, the removable base being removably adhered to the frame layer base portion.

In certain embodiments, the film assembly can include a transfer film that is removably adhered to the removable adhesion layer.

In some embodiments, the ink layer can include a non-toxic ink.

In certain embodiments, the transfer layer can be formed from resilient materials.

In some embodiments, the surface can be human skin

The present invention is also directed toward a film assembly system including a film assembly array.

In some embodiments, the film assembly array can include a plurality of film assemblies.

In certain embodiments, the film assembly array can be disposed within an assembly housing.

In various embodiments, the plurality of film assemblies can include the film assembly.

The present invention is also directed toward a method for manufacturing a film assembly. In various embodiments, the method can include one or more of the steps of removably adhering an ink layer to a base layer, removably adhering a developer layer to the ink layer, and removably adhering a transfer layer to the developer layer so that an image of the transfer layer can be transferred to a surface.

In some embodiments, the method for manufacturing the film assembly can include the step of removably adhering a frame layer to the transfer layer.

In certain embodiments, the method for manufacturing the film assembly can include the step of removably adhering a transfer film to the frame layer.

In some embodiments, the frame layer can include a removable base and a frame layer base portion that is removably adhered to the removable base.

In various embodiments, the ink layer of the film assembly can include a non-toxic ink.

In certain embodiments, the base layer and the ink layer can be adhered together to form a rear layer

In various embodiments, the transfer layer can be translucent.

In some embodiments, the present invention is also directed toward a film assembly for transferring an image to a surface, the film assembly including a removable rear layer having a back base layer and an ink layer, a first adhesion layer that is removably adhered to the removable rear layer, a developer layer that is removably adhered to the first adhesion layer, a first transfer layer that is removably adhered to the developer layer, the first transfer layer including the image that is configured to be transferred onto the surface, and a frame layer that is removably adhered to the first transfer layer, the frame layer being configured to be separated from the first transfer layer.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic side view of a film assembly having features of the present invention in accordance with various embodiments herein;

FIG. 2 is an exploded perspective view of one embodiment of a portion of the film assembly;

FIG. 3A is a side view of one embodiment of the film assembly;

FIG. 3B is a simplified close-up side view of the film assembly illustrated in FIG. 3A;

FIG. 4 is a front view of one embodiment of a portion of the film assembly including a frame, the film assembly including a fully developed image and part of the frame detached from the fully developed image;

FIG. 5 is a rear view of one embodiment of a portion of the film assembly with a portion of the rear layer removed from the developer layer;

FIG. 6 is a front view of one embodiment of a portion of the film assembly system including a surface illustrated as human skin;

FIG. 7 is a perspective view of one embodiment of a film assembly system including an assembly housing illustrated in phantom and a film assembly array including a plurality of the film assemblies;

FIG. 8 is a flow chart of one embodiment of a method for manufacturing the film assembly having steps of the present invention;

FIG. 9 is a flow chart of one embodiment of a method for transferring an image from the film assembly to a surface having steps of the present invention; and

FIG. 10 is a flow chart of another embodiment of a method for transferring an image from the film assembly to the surface.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DESCRIPTION

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

As an overview, in various embodiments, the film assemblies and methods of manufacture are disclosed herein describe the use of film assemblies including any number of layers for manufacturing the film assemblies for attaching an image to a surface. The film assemblies herein can include one or more of a base layer(s), an ink layer that is removably adhered to the base layer, a developer layer that develops the ink layer, and a transfer layer that is removably adhered to the developer layer and is configured to be transferred onto the surface. It is appreciated that the film assemblies herein can include many different forms. The film assemblies described herein receive a captured image from a camera or another image capturing device and self-develop the image for removal and placement onto a surface.

As used herein, the terms “film assembly” and “film” are sometimes used interchangeably unless otherwise noted. Also, as used herein, the terms “frame” and “frame layer” are sometimes used interchangeably unless otherwise noted.

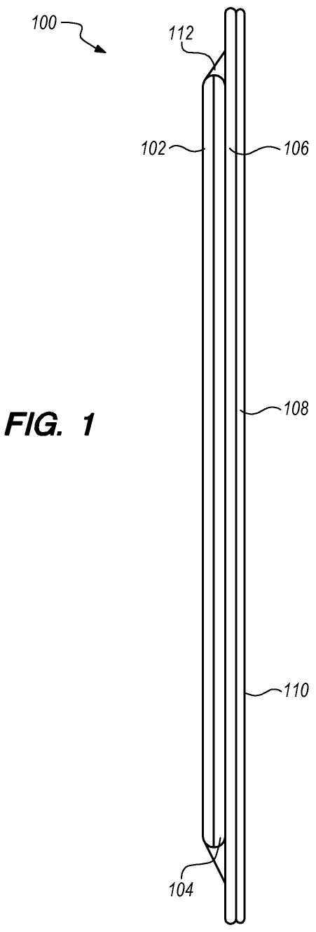

FIG. 1 is a simplified schematic side view of a film assembly 100. The film assembly 100 is suitable for attaching an image that is captured using an image capture device (not shown) to a surface. As used herein, the term “camera” means any image capture device, instant film camera, cellular telephone camera, image capture system, or device for capturing an image. Also, as used herein, the term “capture” means taking an image, snapping a photograph, exposing ink layers to light, or any method for creating an image using an image capture device. In some embodiments, the camera can be an instant film capture device that can eject an instant film assembly. In other embodiments, the film assembly 100 can be used as a standard instant film developed image. It is further recognized that the structures included in FIG. 1 (as well as other figures shown and described herein) are not necessarily drawn to scale for ease of viewing and/or understanding.

The size, shape, configuration, and design of the film assembly 100 can vary. In the embodiment illustrated in FIG. 1, the film assembly 100 can include one or more base layers 102, one or more ink layers 104, one or more removable adhesion layers 112, one or more developer layers 106, a transfer layer 108, and a transfer film 110.

The base layer 102 is suitable for protecting the ink layer and other layers from outside forces and elements. In some embodiments, the base layer 102 can be flexible. The size, shape, and the design of the base layer 102 can vary depending on the design requirements of the base layer 102 and film assembly 100. The base layer 102 can be formed from suitable materials including papers, plastics, metals, cardboards, adhesives, or any other suitable materials. The base layer 102 can be removably attached to the ink layer 104. In other embodiments, the base layer 102 can be bonded together with ink layer 104 and wrapped in a housing.

The ink layer 104 can be a single layer or in other embodiments, can include a plurality of ink layers. The ink layer 104 can include inks, dyes, or other chemicals that are light sensitive. Depending on the dye, ink, or other chemical used, the ink layer 104 can be sensitive to a different wavelength of light and can correspond to a certain visible color wavelength. The ink layer 104 works in combination with the developer layer 108 to develop an image onto the transfer layer 108. The size, shape, and the design of the ink layer 104 can vary depending on the design requirements of the ink layer 104 and film assembly 100. The ink layer 104 can be formed from suitable materials including papers, plastics, metals, cardboards, chemicals, adhesives, or any other suitable materials. The ink layer 104 can be removably attached to the base layer 102 and the developer layer 106.

The developer layer 106 can be a single layer or in other embodiments, can include a plurality of developer layers. After an image is captured, the developer layer 106 works in combination with the ink layer 104 to develop an image in real-time onto the transfer layer 108. The developer layer 108 can use chemical development, digital development, or any other suitable development methods. In non-exclusive embodiments, chemicals used in film development of the transfer layer can include hydroquinone, phenindione, dimezone, sodium carbonate and/or sodium hydroxide. It is appreciated that other chemicals may be used to achieve the same or similar effects. In certain embodiments, the developer layer 106 will be directly applied to a surface to apply the developed image. In certain embodiments, the developer layer 106 can be made from resilient materials to conform to the unevenness of any surface. In other embodiments, the developer layer 106 can be removed from the film assembly 100. The size, shape, and the design of the developer layer 106 can vary depending on the design requirements of the developer layer 106 and film assembly 100. The developer layer 106 can be formed from suitable materials including papers, plastics, metals, cardboards, chemicals, adhesives, or any other suitable materials. The developer layer 106 can be removably attached to the ink layer 104 and the transfer layer 108. In non-exclusive embodiments, the developer layer 106 can be removably adhered to a removable adhesion layer 112 and the transfer layer 108.

The transfer layer 108 is the layer that will be applied to a surface. The transfer layer 108 is the layer which contains the fully developed image. The size, shape, and the design of the transfer layer 108 can vary depending on the design requirements of the transfer layer 108 and film assembly 100. In some embodiments, the transfer layer can have a removable frame around the developed image. In non-exclusive embodiments, a frame layer can be removably adhered to the transfer layer 108 and the frame layer can be removed from the transfer layer 108 before transferring the image to a surface. In certain embodiments, the transfer layer can have a removable base. The transfer layer 108 can be formed from suitable materials including papers, plastics, metals, cardboards, adhesives, or any other suitable materials. In non-exclusive embodiments, the transfer layer 108 can be translucent and flexible. In certain embodiments, the transfer film 110 can be made from resilient materials to conform to the unevenness of any surface. The transfer layer 108 can be removably attached to the developer layer 106 and transfer film 110.

The transfer film 110 is a layer that can be used to protect the transfer layer 108 and image from outside forces and elements. In particular, the transfer film 110 is made to support the transfer layer 108 while a wet cloth is held on the transfer film. The transfer film 110 can prevent the image from being transferred to the wet cloth instead of the desired surface. The transfer film 110 can be formed from suitable materials including papers, plastics, metals, cardboards, adhesives, or any other suitable materials. In certain embodiments, the transfer film 110 can be made from resilient materials to conform to the unevenness of any surface. The transfer film 110 can be removably attached to the transfer layer 108. In some embodiments, the transfer film 110 can be configured to peel off the transfer layer 108 after the image has been applied to a surface.

The removable adhesion layer 112 is a layer that can be used to removably attach the various layers of film assembly 100 together. In some embodiments, the removably adhesion layer 112 can be inserted in between the ink layer 104 and developer layer 104. The removable adhesion layer 104 can be attached to create an adhesive side and an opposed non-adhesive side for any of the various layers of the film assembly 100. The removable adhesion layer 112 can be formed from suitable materials including papers, plastics, metals, cardboards, adhesives, or any other suitable materials. In certain embodiments, the removable adhesion layer 112 can be made from resilient materials to conform to the unevenness of any given layer. In some embodiments, the removably adhesion layer 112 can be configured to peel off the various layers or can be configured to be adhered in a non-permanent manner. In other embodiments, the removable adhesion layer can permanently bind the layers of film assembly 100 together.

As with all embodiments illustrated and described herein, various structures may be omitted from the figures for clarity and ease of understanding. Further, the figures may include certain structures that can be omitted without deviating from the intent and scope of the invention.

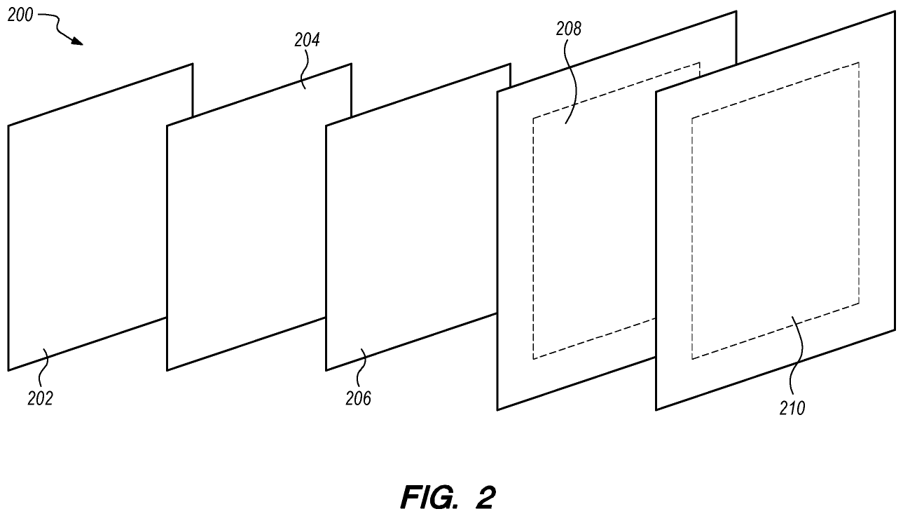

FIG. 2 is an exploded front perspective view of one embodiment of a portion of the film assembly 200. In the embodiment illustrated in FIG. 2, the film assembly 200 has been simplified and can include one or more layers. The size, shape, configuration, and design of the film assembly 200 can vary. In the embodiment illustrated in FIG. 2, the film assembly 200 can include one or more base layers 202, one or more ink layers 204, one or more developer layers 206, a transfer layer 208, and a transfer film 210. The one or more base layers 202, one or more ink layers 204, one or more developer layers 206, a transfer layer 208, and a transfer film 210 can be substantially similar to the layers previously shown and described herein and/or shown in greater detail below.

As demonstrated in FIG. 2, the transfer layer 208 and the transfer film 210 can be larger in size than the base layer 202, the one or more ink layers 204 and the one or more developer layers 206. In some embodiments, the transfer layer 208 and the transfer film 210 can have markings demonstrated with dashed lines to indicate where the base layer 202, the one or more ink layers 204 and the one or more developer layers 206 attach to the transfer layer 208. In non-exclusive embodiments, the frame layer (integrated with the transfer layer 208 as shown by the dashed lines) can be removably adhered to the transfer layer 208 and the frame layer can be removed from the transfer layer 208 before transferring the image to a surface. Still alternatively, the transfer layer 208 and the transfer film 210 can have their middle portions removed along the dashed line to expose the developed image of the transfer layer 208.

The layers in film assembly 200 can be formed from various materials and include differing compositions. For example, and in some embodiments, the ink layer 204 can contain a non-toxic ink. In certain embodiments, the transfer layer 208 can be formed from translucent materials. In various embodiments, the transfer film 210 can be formed from paper materials. Still alternatively, the base layer 202, the ink layer 204, the developer layer 206, the transfer layer 208, and the transfer film 210 can be formed from any other suitable material(s). As provided in greater detail herein, the geometry, configuration, size and/or shape of the layers of film assembly 200 can also be varied to suit the design requirements of the film assembly 200.

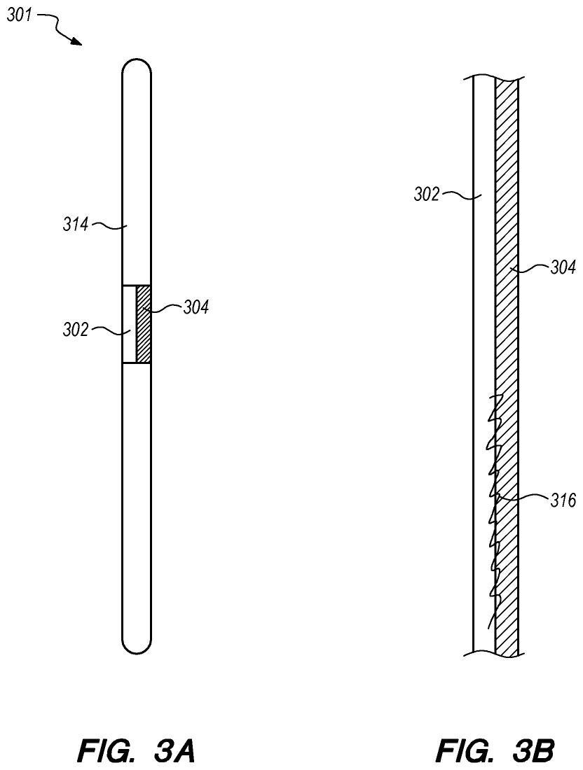

FIG. 3A is a side view of the portion of the film assembly of one embodiment of a portion of the film assembly, with a portion of the rear layer 301 exposed, showing the interior components of the rear layer 301. In this embodiment of a portion of the film assembly, the rear layer 301 includes a rear layer housing 314, one or more base layers 302, and one or more ink layers 304. The base layer 302 and ink layer 304 can be substantially similar to the base layers and ink layers previously shown and described herein and/or shown in greater detail below.

The rear layer 301 can serve the function of protecting the inner layers, base layer 302 and ink layer 304. In other embodiments, the rear layer 301 can only include one or more base layers 302 and no ink layers. Upon applying the image to a surface, the ink layer 304 can remain in the film assembly during transfer. Removal of the ink layer 304 can minimize the amount of ink left on the surface. The rear layer 301 can also have an adhesive side and an opposed non-adhesive side for ease of removal from the film assembly 100. The rear layer 301 can be connected to the developer layer of the film assembly previously shown and described herein and/or shown in greater detail below.

In the embodiment illustrated in FIG. 3A, the rear layer housing 314 can be formed from suitable materials including papers, plastics, metals, cardboards, adhesives, or any other suitable material(s). The size, shape, configuration, and the design of the rear layer housing 314 can vary depending on the design requirements of the rear layer housing 314.

FIG. 3B is a simplified schematic side view of one embodiment of a portion of the film assembly, including another embodiment of a portion of the film assembly, showing the interior components of the portion of the film assembly illustrated in FIG. 3A.

Inside of the rear layer housing 314, one or more base layers 302 and one or more ink layers 304 can be bound together using an adhesion layer 316. In this embodiment, the adhesion layer 316 is shown and can adhere base layer 302 to ink layer 304, thereby increasing the ease of removal of the entire rear layer 301 from the developer layer. The adhesion layer 316 can be substantially similar to the removable adhesion layers previously shown and described herein and/or shown in greater detail below. As provided in greater detail herein, the geometry, configuration, size and/or shape of the adhesion layer 316 can also be varied to suit the design requirements of the rear layer 301.

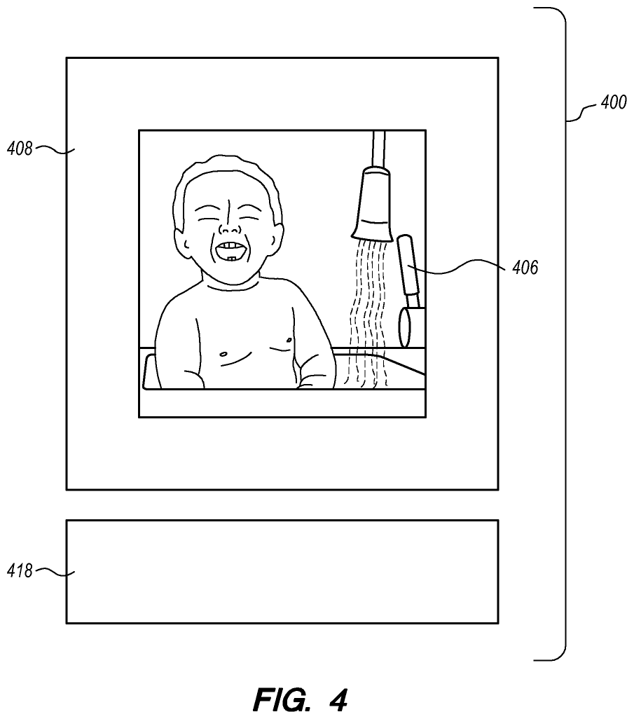

FIG. 4 is a front view of one embodiment of the film assembly 400 with the developer layer 406 and the transfer layer 408. In the embodiment illustrated in FIG. 4, the transfer layer 408 includes a removable base 418, the removable base 418 being removably adhered to the transfer layer 408. In certain embodiments, the removable base 418 can be removed in order to reduce the amount of transfer film applied to the surface.

In various embodiments, the removable base 418 and the transfer layer 408 can include indicated perforations, tear seams, or other variations. In some embodiments, the film assembly 400 can be pulled out of an image capture device by the removable base 418 without becoming detached from the transfer layer 408. Additionally, or in the alternative, the transfer layer 408 can be removed from the developer layer 406. The removable base 418 can be formed from suitable materials including papers, plastics, metals, cardboards, adhesives, or any other suitable material(s). The size, shape, configuration, and the design of the rear layer the removable base 418 can vary depending on the design requirements of the film assembly 400, the transfer layer 408, or the developer layer 406.

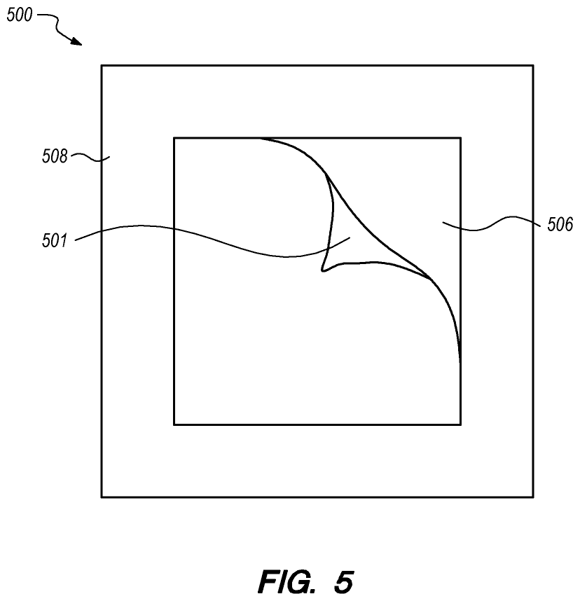

FIG. 5 is a rear view of one embodiment of a portion of the film assembly 500 with a portion of the rear layer 501 removed. In the embodiment illustrated in FIG. 5, the rear layer 501 is peeled back to reveal the adhesive side that can be adhered to the developer layer 506. In this embodiment, the rear layer 501 can be completed removed from the film assembly 500 to expose the developer layer 506. Additionally, or in the alternative, a portion of the transfer layer 508 can be removed from the film assembly 500. After the rear layer 501 has been removed, the film assembly 500 can apply the image to a surface (to be demonstrated in later figures, hereinafter).

The operation and function of the film assembly 500 can be substantially similar to those previously described. However, in this embodiment, the rear layer 501 can be completely removed from the film assembly 500 to expose the developer layer 506. In one embodiment, the developer layer 506 can come in direct contact with the surface on which the image is to be applied. As mentioned above, in some embodiments, a frame layer (shown as integrated with transfer layer 508 in FIG. 5) can be removably adhered to the transfer layer 508 and the frame layer can be removed from the transfer layer 508 before transferring the image to a surface. In non-exclusive embodiments, the developer layer 506 can be removed from the transfer layer 508 after the developer layer 506 has developed an image onto the transfer layer 508.

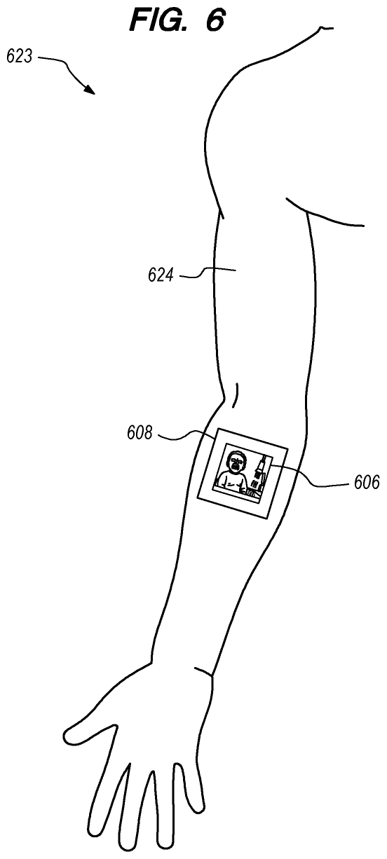

FIG. 6 is a front view of one embodiment of a portion of the film assembly system 623 including a surface 624 illustrated as human skin. The surface 624 can be human skin, animal skin, walls, vehicles, electronics, furniture, or any other suitable surface(s) to which an image can be attached. In the embodiment illustrated in FIG. 6, the transfer layer 608 is applied to the surface 624 with developer layer 606 on the opposed side of the portion of the film assembly system 623. In non-exclusive embodiments, the transfer layer can be applied to the surface 624 via an adhesive or adhesive layer. In other embodiments, the transfer layer 608 is applied to a surface using human-safe ink and pressing the film assembly system 623 with a wet cloth for a set duration of time. It is appreciated that the film assembly system 623 can apply an image to the surface 624 using any suitable method(s).

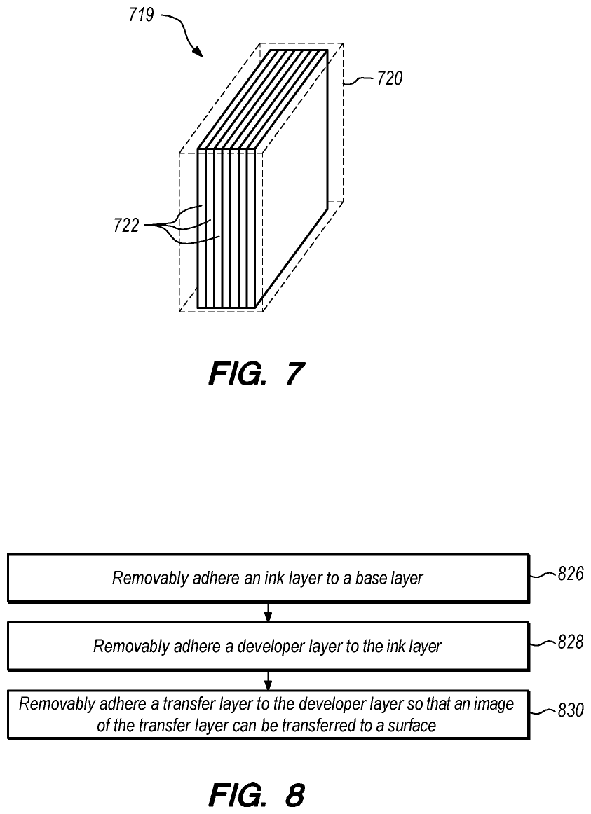

FIG. 7 is a perspective view of one embodiment of the film assembly system 719 including an assembly housing 720 illustrated in phantom and a film assembly array 722, the film assembly array including a plurality of the film assemblies. In the embodiment illustrated in FIG. 7, the film assembly array can include the film assembly demonstrated in FIGS. 1 through 5 above, but can also include other film assemblies. The assembly housing 720 can be formed from suitable materials including papers, plastics, metals, cardboards, adhesives, or any other suitable material(s). Additionally, or in the alternative, the film assembly housing 702 can be opened via tearing, cutting, other handheld opening methods, or any other suitable method(s). The film assembly array 722 can be arranged so that each individual film assembly within the assembly array 722 can be removed from the array 722.

FIG. 8 is a flow chart showing steps of one embodiment of a method for manufacturing a film assembly having features and/or steps of the present invention. It is understood that the method pursuant to the disclosure herein can include greater or fewer steps than those shown and described relative to FIG. 8. Stated another way, the method according to the present invention can omit one or more steps illustrated in FIG. 8 or can add additional steps not shown and described in FIG. 8, and still fall within the purview of the present invention. Further, the sequence of the steps can be varied from those shown and described relative to FIG. 8. The sequence of steps illustrated in FIG. 8 is not intended to limit the sequencing of steps in any manner.

In the embodiment illustrated in FIG. 8, at step 826, an ink layer is removably adhered to a base layer. The base layer and ink layer can be constructed and/or can include components, chemicals, etc. as previously described herein, but can also include other base layer(s) and ink layer(s). In some embodiments, an adhesion layer can be used to removably adhere the base layer to the ink layer. In non-exclusive embodiments, the base layers and ink layers can have adhesive sides and opposed non-adhesive sides. Alternatively, the base layer and ink layer can include additional or alternative components provided the base layer provides a protective layer and the ink layer contains ink, components, or chemicals needed to create an image.

At step 828, a developer layer is removably adhered to the ink layer. The developer layer can be constructed and/or can include components, chemicals, etc. as previously described herein, but can also include other developer layer(s). In some embodiments, an adhesion layer can be used to removably adhere the developer layer to the ink layer. In non-exclusive embodiments, the developer layer can have an adhesive side and an opposed non-adhesive side. Alternatively, the developer layer can include additional or alternative components provided the developer layer utilizes development chemicals, components, or other viable development method(s).

At step 830, a transfer layer is removably adhered to the developer layer so that an image of the transfer layer can be transferred to a surface. The transfer layer can be constructed and/or can include components, chemicals, etc. as previously described herein, but can also include other transfer layer(s). In some embodiments, an adhesion layer can be used to removably adhere the developer layer to the transfer layer. In non-exclusive embodiments, transfer layer can have an adhesive side and an opposed non-adhesive side. Alternatively, the transfer layer can include additional or alternative components provided the transfer layer is configured to transfer the developed image onto a surface.

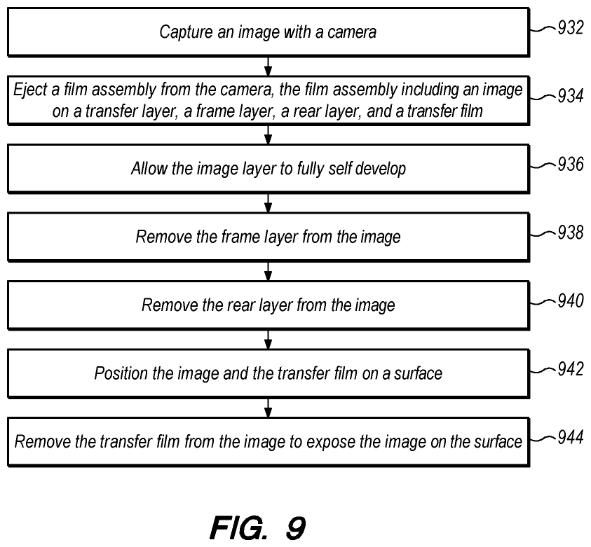

FIG. 9 is a flow chart of one embodiment of a method for transferring an image from a film assembly to a surface. It is understood that the method pursuant to the disclosure herein can include greater or fewer steps than those shown and described relative to FIG. 9. Stated another way, the method according to the present invention can omit one or more steps illustrated in FIG. 9 or can add additional steps not shown and described in FIG. 9, and still fall within the purview of the present invention. Further, the sequence of the steps can be varied from those shown and described relative to FIG. 9. The sequence of steps illustrated in FIG. 9 is not intended to limit the sequencing of steps in any manner.

In the embodiment illustrated in FIG. 9, at step 932, an image is captured with a camera. The image is captured by the camera using any suitable method(s). In one embodiment, the camera can be constructed and/or can include components as previously described herein. Alternatively, the camera herein described can include additional or alternative components provided the camera is configured to capture an image.

At step 934, a film assembly is ejected from the camera, the film assembly including an image on a transfer layer, a frame layer, a rear layer, and a transfer film. The film assembly can be constructed and/or can include components as previously described herein. The film assembly in step 934 includes the undeveloped image captured by the camera in step 932. The film assembly can be ejected from the camera by pulling or grabbing the frame layer, transfer layer, or any other suitable layer(s) that extrude from the camera after the image is taken. Alternatively, the camera can self-eject the film assembly using suitable method(s).

At step 936, the image is allowed to fully self-develop. The image can be constructed and/or can include components as previously described herein. The image can self-develop using development method(s) previously described herein. Alternatively, the image can include additional or alternative components provided the image is configured to self-develop.

At step 938, the frame layer is removed from the image. The frame layer(s) can be constructed and/or can include components as previously described herein. Alternatively, the frame layer can include additional or alternative components provided the frame layer is configured to be removable from the image. The image and frame layer can be separated using indicated perforations, tear seams, or other variations. In non-exclusive embodiments, the image and frame layer can be separated using any viable method(s).

At step 940, a rear layer is removed from the image. As used herein, the term “rear layer” means any back structural layer, but can also include only a single base layer, such as the previously described back base layer or developer layer. The rear layer(s) can be constructed and/or can include components as previously described herein. In one embodiment, the rear layer can include the rear layer demonstrated in FIGS. 3A and 3B. Alternatively, the rear layer can include additional or alternative components provided the rear layer is configured to be removable from the image, the transfer layer or the developer layer.

At step 942, the transfer film and image are positioned to a surface. The surface can be any surface as previously described herein, but can be human skin, animal skin, walls, vehicles, electronics, furniture, or any other suitable surface(s) to which an image can be attached. In one embodiment, the surface can include the human arm demonstrated in FIG. 7.

The image can be positioned on a surface using a variety of methods. In certain embodiments, the image is held with a wet cloth for a set period. In one embodiment, the image is held with a wet cloth for at least 30 seconds. Durations of holding the image with the wet cloth can vary depending on the type of film being used, the design requirements of the transfer layer, and other considerations. The wet cloth can include common kitchen towels, bath towels, cotton towels, used t-shirts, or any other suitable cloth configured to retain water.

After the image has been positioned onto a surface, the wet cloth is removed and the transfer film is removed. Upon removal of the wet cloth and transfer film, the image should properly be attached to the surface. In some embodiments, if the image is not fully attached upon removal of the wet cloth, the user can reapply the transfer film and the wet cloth for an additional period of time.

At step 944, a transfer film is removed to expose the image on the surface. The transfer film can be constructed and/or can include components as previously described herein. Alternatively, the transfer film can include additional or alternative components provided the transfer film is configured to protect the transfer layer and image from outside forces and elements. The transfer film can have an adhesive or adhesive layer on the side facing the transfer film.

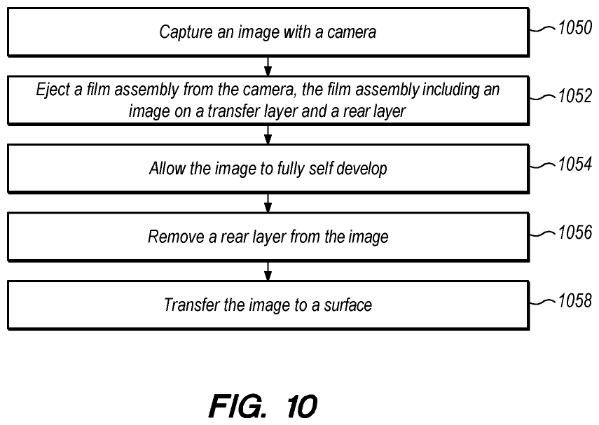

FIG. 10 a flow chart of one embodiment of a method for transferring an image from a film assembly to a surface. It is understood that the method pursuant to the disclosure herein can include greater or fewer steps than those shown and described relative to FIG. 10. Stated another way, the method according to the present invention can omit one or more steps illustrated in FIG. 10, or can add additional steps not shown and described in FIG. 10, and still fall within the purview of the present invention. Further, the sequence of the steps can be varied from those shown and described relative to FIG. 10. The sequence of steps illustrated in FIG. 10 is not intended to limit the sequencing of steps in any manner.

In the embodiment illustrated in FIG. 10, at step 1050, an image is captured with a camera. The image is captured by the camera using any suitable method(s). In one embodiment, the camera can be constructed and/or can include components as previously described herein. Alternatively, the camera herein described can include additional or alternative components provided the camera is configured to capture an image.

At step 1052, a film assembly is ejected from the camera, the film assembly including an image on a transfer layer and a rear layer. The film assembly can be constructed and/or can include components as previously described herein. The film assembly in step 1052 includes the undeveloped image captured by the camera in step 1050. The film assembly can be ejected from the camera by pulling or grabbing the frame layer, transfer layer, or any other suitable layer(s) that extrude from the camera after the image is taken. Alternatively, the camera can self-eject the film assembly using suitable method(s).

At step 1054, the image is allowed to fully self-develop. The image can be constructed and/or can include components as previously described herein. The image can self-develop using development method(s) previously described herein. Alternatively, the image can include additional or alternative components provided the image is configured to self-develop.

At step 1056, a rear layer is removed from the image. The rear layer(s) can be constructed and/or can include components as previously described herein. In one embodiment, the rear layer can include the rear layer demonstrated in FIGS. 3A and 3B. Alternatively, the rear layer can include additional or alternative components provided the rear layer is configured to be removable from the image, the transfer layer or developer layer.

At step 1058, the image is transferred to a surface. In one embodiment, the image from the transfer layer is adhered to the surface using an adhesive and/or adhesive layer. The image can be transferred by method(s) as previously described herein. Alternatively, the transfer method(s) can include additional or alternative steps provided image transferred to a surface.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It is recognized that the figures shown and described are not necessarily drawn to scale, and that they are provided for ease of reference and understanding, and relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the film assemblies have been illustrated and described herein, one or more features of any one embodiment can be combined with one or more features of one or more of the other embodiments, provided that such combination satisfies the intent of the present invention.

While a number of exemplary aspects and embodiments of the film assemblies have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A film assembly for transferring an image to a surface, the film assembly comprising:
a base layer;
an ink layer that is removably adhered to the base layer;
a developer layer that develops the ink layer; and
a transfer layer that is removably adhered to the developer layer, the transfer layer including the image that is configured to be transferred onto the surface.

2. The film assembly of claim 1 wherein the base layer and the ink layer are adhered together to form a rear layer.

3. The film assembly of claim 2 wherein the rear layer has an adhesive side and an opposed non-adhesive side.

4. The film assembly of claim 2 wherein the rear layer is removable from the developer layer.

5. The film assembly of claim 1 wherein the transfer layer includes a frame layer with a frame layer base portion, the frame layer being removably adhered to the transfer layer.

6. The film assembly of claim 5 wherein the frame layer includes a removable base, the removable base being removably adhered to the frame layer base portion.

7. The film assembly of claim 1 further comprising a transfer film that is removably adhered to the transfer layer.

8. The film assembly of claim 1 wherein the ink layer contains a non-toxic ink.

9. The film assembly of claim 1 wherein the transfer layer is formed from resilient materials.

10. The film assembly of claim 1 wherein the surface is human skin.

11. A film assembly system including a film assembly array, the film assembly array including a plurality of film assemblies being disposed within an assembly housing, the plurality of film assemblies including the film assembly of claim 1.

12. A method for manufacturing a film assembly, the method comprising the steps of:
removably adhering an ink layer to a base layer;
removably adhering a developer layer to the ink layer; and
removably adhering a transfer layer to the developer layer so that an image of the transfer layer can be transferred to a surface.

13. The method of claim 12 further comprising the step of removably adhering a frame layer to the transfer layer.

14. The method of claim 13 further comprising the step of removably adhering a transfer film to the frame layer.

15. The method of claim 13 wherein the frame layer includes a removable base and a frame layer base portion that is removably adhered to the removable base.

16. The method of claim 12 wherein the ink layer of the film assembly contains a non-toxic ink.

17. The method of claim 12 wherein the transfer layer of the film assembly is manufactured with resilient materials.

18. The method of claim 12 wherein the base layer and the ink layer are adhered together to form a rear layer.

19. The method of claim 12 wherein the transfer layer is translucent.

20. A film assembly for transferring an image to a surface, the film assembly comprising:
a removable rear layer having a base layer and an ink layer;
a first adhesion layer that is removably adhered to the removable rear layer;
a developer layer that is removably adhered to the first adhesion layer;
a first transfer layer that is removably adhered to the developer layer, the first transfer layer including the image that is configured to be transferred onto the surface; and
a frame layer that is removably adhered to the first transfer layer, the frame layer being configured to be separated from the first transfer layer.
